(19) United States
(12) Patent Application Publication
(10) Pub. No.: US 2007/0042953 A1
(43) Pub. Date: Feb. 22, 2007

(54) Antiepileptogenic complex of albumin with docosahexaenoate

(76) Inventors: Nicolas G. Bazan, New Orleans, LA (US); Alberto E. Musto, Covington, LA (US); Victor L. Marcheselli, Covington, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 11/253,483

(22) Filed: Oct. 18, 2005

Related U.S. Application Data
(60) Provisional application No. 60/708,439, filed on Aug. 16, 2005.

(51) Int. Cl. A61K 38/26; A61K 31/202

(52) U.S. Cl. 514/12; 514/560

(57) ABSTRACT

The infusion of an albumin-docosahexaenoic acid (DHA) complex was shown to inhibit the progression of kindling epileptogenesis. This was shown in mice using kindling as the experimental epilepsy model. The DHA-albumin complex was shown to affect the activity of the brain when administered intraperitoneally. This therapy could also be administered intravenously. This therapy would also be effective against chronic epilepsy.

The development of this invention was partially funded by the United States Government under grant NS23002, from the National Institutes of Health. The United States Government has certain rights in this invention.

This invention pertains to a method to treat or ameliorate epileptogenesis or chronic epilepsy by administering a complex of albumin and docosahexaenoic acid.

The omega-3 fatty acid docosahexaenoic acid (22:6, n-3, DHA) is highly concentrated in synapses, is required during development and for synaptic plasticity, and participates in neuroprotection. Free DHA is released through phospholipases from membrane phospholipids in response to seizures. See, N. G. Bazan, “Effects of ischemia and electroconvulsive shock on free fatty acid pool in the brain,” Biochim. Biophys. Acta, vol. 218, pp. 1-10 (1970); and D. L. Birkle et al., “Effect of bicuculline-induced status epilepticus on prostaglandins and hydroxyeicosatetraenoic acids in rat brain subcellular fractions,” J. Neurochem., vol. 48, pp. 1768-1778 (1987). Recently the structure and bioactivity of neuroprotectin D1, a potent DHA-derived mediator in brain ischemia-reperfusion and in oxidative stress, has been described. See V. L. Marcheselli et al., “Novel docosanoids inhibit brain ischemia-reperfusion-mediated leukocyte infiltration and pro-inflammatory gene expression,” J. Biol. Chem., vol. 278, pp. 43807-817 (2003); and P. K. Mukherjee et al., “Neuroprotectin D1: A docosahexaenoic acid-derived docosatriene protects human retinal pigment epithelial cells from oxidative stress,” Proc. Natl. Acad. Sci., USA, vol. 101, pp. 8491-96 (2004). Several polyunsaturated fatty acids (PUFAs), including DHA, have been suggested to attenuate epileptic activity in in vitro studies on rat brain cells or hippocampal slices. See C. Young et al., “Docosahexaenoic acid inhibits synaptic transmission and epileptiform activity in the rat hippocampus,” Synapse, vol. 37, pp. 90-94 (2000); and Y. Xiao et al., “Polyunsaturated fatty acids modify mouse hippocampal neuronal excitability during excitotoxic or convulsant stimulation,” Brain Res., vol. 846, pp. 112-121 (1999). In addition, nutritional supplementation with DHA was found to reduce seizure frequency in the first 6 weeks, but the effect was not sustained. See A. W. Yuen et al., “Omega-3 fatty acid supplementation in patients with chronic epilepsy: A randomized trial,” Epilepsy Behav., vol. (Epub; Jul. 7, 2005; ahead of print).

DHA complexed to albumin has been shown to enhance neuroprotectin 1 synthesis in human retinal pigment epithelial cells, and to be strongly neuroprotective in a mouse model of brain ischemia. See P. K. Mukherjee et al., “Neuroprotectin D1: A docosahexaenoic acid-derived docosatriene protects human retinal pigment epithelial cells from oxidative stress,” Proc. Natl. Acad. Sci., U.S.A., vol. 101, pp. 8491-8496 (2004); and L. Belayev et al., “Docosahexaenoic acid complexed to albumin elicits high-grade ischemic neuroprotection,” Stroke, vol. 36, pp. 118-123 (2005).

Kindling is an experimental epilepsy model in which repeated electrical stimulation of certain forebrain structures will trigger progressively more intense electroencephalogenic and behavioral seizure activity. This activity, once established, results in a permanent state of susceptibility to seizures, including spontaneous seizures. See B. Adams et al., “Nerve growth factor accelerates seizure development, enhances mossy fiber sprouting, and attenuates seizure-induced decreases in neuronal density in the kindling model of epilepsy,” J. Neuroscience, vol. 17, pp. 5288-5296 (1997). The use and characterization of the kindled state has been important in gaining insight into the pathology and potential treatment of epilepsy.

We have discovered that the administration of an albumin-docosahexaenoic acid (DHA) complex has an inhibitory effect on the early stages of epileptogenesis. This was shown in mice using kindling as the experimental epilepsy model. The DHA-albumin complex was shown to affect the activity of the brain when administered intraperitoneally. This therapy could also be administered intravenously, and could also be effective against chronic epilepsy.

### BRIEF DESCRIPTION OF DRAWINGS

FIG. 1a illustrates the difference in Racine's Score, a behavioral score of epilepsy, in two groups of mice, one group infused with albumin only and one group infused with the DHA-albumin complex.

FIG. 1b illustrates the difference in measured electrical events in two groups of mice, one group infused with albumin only and one group infused with the DHA-albumin complex.

FIG. 1c illustrates the difference in measured epileptic events in two groups of mice, one group infused with albumin only and one group infused with the DHA-albumin complex.

FIG. 1d illustrates the difference in electrical events, expressed as the power spectral density, in two groups of mice, one group infused with albumin only and one group infused with the DHA-albumin complex.

FIG. 1e illustrates the difference in Racine's Score, a behavioral score of epilepsy, in two groups of mice, one group infused with albumin only and one group infused with the DHA-albumin complex at day 2.5 of the kindling procedure.

FIG. 2a illustrates representative membrane potential responses recorded in pyramidal neurons from mice treated in vivo with albumin or with the DHA-albumin complex during injections of various currents (−100, −30, 10, 30 and 150 pA).

FIG. 2b illustrates the firing frequency (Hz) as a function of the injected current as measured in murine hippocampal CA1 pyramidal neurons in two groups of mice, one group infused with only albumin and one group infused with the DHA-albumin complex.

FIG. 3 illustrates the amount of 10,17S-docosatriene (neuroprotectin D1) from various regions of the brain during kindling, both from endogenous DHA (Endogenous) and from labeled infused DHA (d5).

### EXAMPLE 1

### Materials and Methods

Preparation of DHA-Albumin Complex

DHA was physically complexed to human albumin by the following method. The complex was prepared under sterile conditions in a laminar-flow hood. DHA (200 mg cis-4,7,10,13,16,19-Docosahexaenoic acid, sodium salt; #D-8768, Simga Co., St. Louis, Mo.) was dissolved in 500 μl ethanol, and then injected into a 100-ml sealed bottle of Buminate 25% (human serum albumin, USP, 25% solution; Baxter Healthcare Corporation, Westlake Village, Calif.). The solution was thoroughly mixed at room temperature in a G24 environmental incubator shaker (New Brunswick Scientific Co., Edison, N.J.) at 500 rpm for 30 min. Quantitative analysis by mass spectrometry and gas-liquid chromatography indicated 1.999 μg DHA/μl solution. The solution was stored at 4° C. protected from light and oxygen (stored under nitrogen). The solution was stable for at least six months.

Kindling Procedure

Male mice (C57BL/6; 20-25 g) were obtained from Charles River Laboratories, Inc. and housed at Louisiana State University Health Sciences Center, Neuroscience Center Animal Care Facilities in accordance with National Institutes of Health guidelines. Protocols were approved by the Louisiana State University Health Sciences Center Institutional Animal Care and Use Committee (IACUC). Tripolar electrode units (Plastic One Inc., Roanoke, Va.) were implanted in the right dorsal hippocampus. After 10 days post surgery, the mice were randomly separated in two groups. Mini-osmotic pumps (Alzet-model 1007D), were prepared and filled with either the DHA-human serum albumin (HSA) complex or with only HSA. The pumps were inserted in the intraperitoneal cavity of each mouse, and an infusion rate of 6.72 μg/kg/day was attained during kindling procedure. The following day kindling was achieved by stimulating 6 times daily for 4 days with subconvulsive electrical stimulation (a 10-sec train containing 50-Hz biphasic pulses of 75-100 μA amplitude) at 30-min intervals. After 1 week another session of stimulation (rekindling) was given. Seizures were graded according to Racine's Scale. An EEG was recorded through electrodes using Enhanced Graphics Acquisition for Analysis (Version 3.63 RS Electronics Inc. Santa Barbara, Calif.) and was analyzed using Neuroexplorer Software (Next Technology).

### EXAMPLE 2

### Infused DHA-Albumin Attenuates Kindling-Induced Epileptogenesis

DHA-albumin when intraperitoneally infused by the implanted minipump produced a marked attenuation of seizures as measured by both Racine's score and epileptic events. Using Racine's score, the behavioral responses were graded on the following scale: grade 1=facial twitches; grade 2=chewing and nodding; grade 3=forelimb clonus; grade 4=rearing, body jerking, tail upholding; and grade 5=imbalance, hind limb clonus, vocalization. The behavioral responses were scored in blind fashion as described by R. J. Racine, “Modification of seizure activity by electrical stimulation. II. Motor seizure,” Electroencephalogr. Clin. Neurophysiol., vol. 32, pp. 281-294 (1972). The mice were also measured for afterdischarge (AD, an EEG measurement of electrical activity). The epileptic events, abnormal electrical signals from the brain (such as spikes, sharp waves, poly-spike-waves, etc. on the EEG) were measured with electrodes using Enhanced Graphics Acquisition for Analysis (Version 3.63, RS Electronics Inc., Santa Barbara, Calif.) during the AD, and were quantified and band frequencies were analyzed using Neuroexplorer Software (Next Technology).

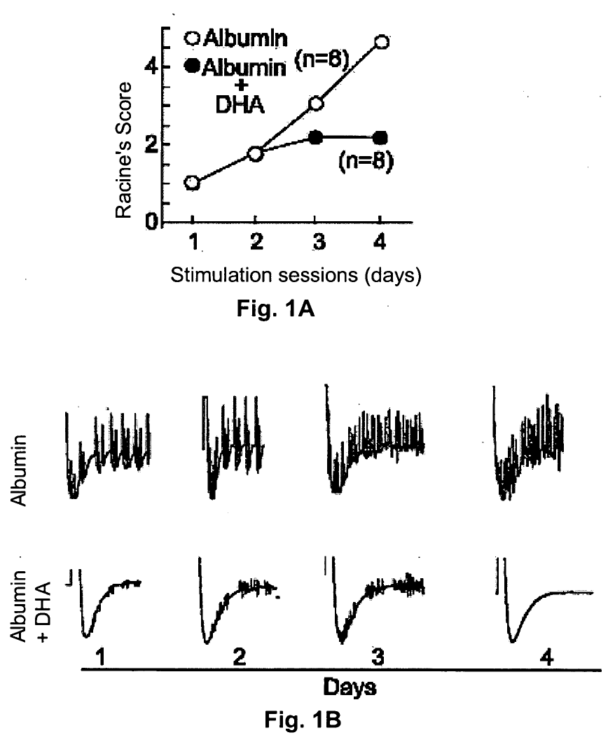

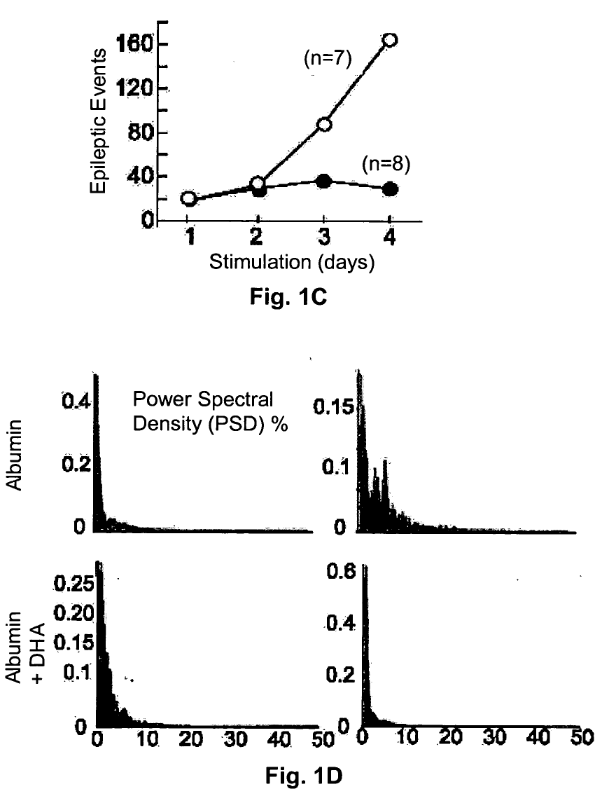

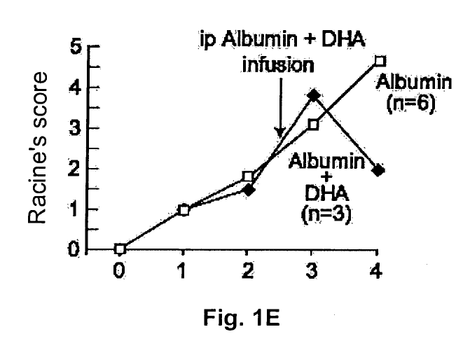

Two groups of mice were intraperitoneally infused by a chronically implanted Alzet minipump during four days of kindling, and infused either with albumin (25%) alone (n=6) or with the albumin DHA complex (n=8). The DHA-albumin complex and albumin were maintained in sterile conditions and were diluted in artificial cerebral spinal fluid (perfusion fluid; Harvard Apparatus, Boston, Mass.). The solutions were stored at 4° C and protected from light and oxygen until use. The delivery rate was 500 nl/hour, 5.8 μg/day/mouse of albumin and 1.68 μg/day/mouse of DHA-albumin. Infusion of the DHA-albumin complex produced a marked attenuation of seizures. (FIG. 1a) In addition, the number of epileptic electroencephalographic events declined. (FIGS. 1b-1d) When DHA-albumin complex was given after day 2 of kindling, attenuation of epileptic events was seen at day 4 of the kindling procedure. (FIG. 1e) These results supported the hypothesis that docosanoids are produced and affect the generation of epileptic events in kindling epileptogenesis.

The DHA-HSA treated group displayed significantly fewer stimulus-evoked motor seizures, and reduced the severity of seizures during kindling as compared with the group receiving only HSA. These observations were correlated with a continuous diminution of the numbers of spikes until the inhibition of the after discharge at the end of the kindling.

### EXAMPLE 3

### DHA-Albumin Reduced Neuronal Membrane Excitability

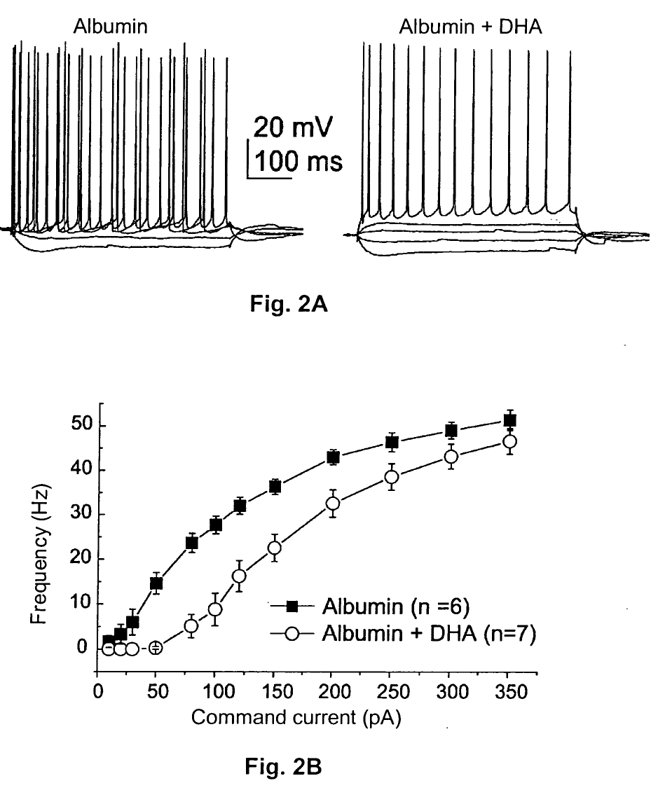

When mice were treated with the DHA-albumin complex, the neuronal membrane excitability was reduced as measured in hippocampal CA1 pyramidal neurons. Two groups of mice were implanted intraperitoneally with Alzet mini-pumps as described above. The mice were infused either with albumin or DHA-albumin as described above. Representative membrane potential responses were recorded in pyramidal neurons from each group during injections of various currents (−100, −30, 10, 30 and 150 pA; see FIG. 2a). The recordings were made using an Axoclamp-2B patch-clamp amplifier in bridge mode as described in C. Chen et al., “Endogenous. PGE2 regulates membrane excitability and synaptic transmission in hippocampal CA1 pyramidal neurons,” J. Neurophysiol., vol. 93, pp. 929-941 (2005). The resting membrane potential for recorded neurons was −62.8±0.9 mV (n=6) for mice treated with albumin, and −66.9±1.0 mV (n=7) for mice treated with DHA-albumin. This difference was statistically significant (p<0.05, one way ANOVA). The membrane input resistance was 112±10.4 MΩ (n=4) in mice treated with albumin, and 85.7±6.3 MΩ(n=7) in mice treated with DHA-albumin complex (“Albumin+DHA” on FIG. 2a), a statistically significant difference (p<0.05, one way ANOVA). FIG. 2b illustrates the firing frequency (Hz) as a function of the injected current as measured in hippocampal CA1 pyramidal neurons in two groups of mice, one group treated with albumin (n=6), and one group treated with the DHA-albumin complex (n=7). These results indicate that the neuronal membrane excitability is reduced in the mice treated with the DHA-albumin complex.

### EXAMPLE 4

### Kindling-Induced Epileptogenesis Triggers Formation of Neuroprotectin D1

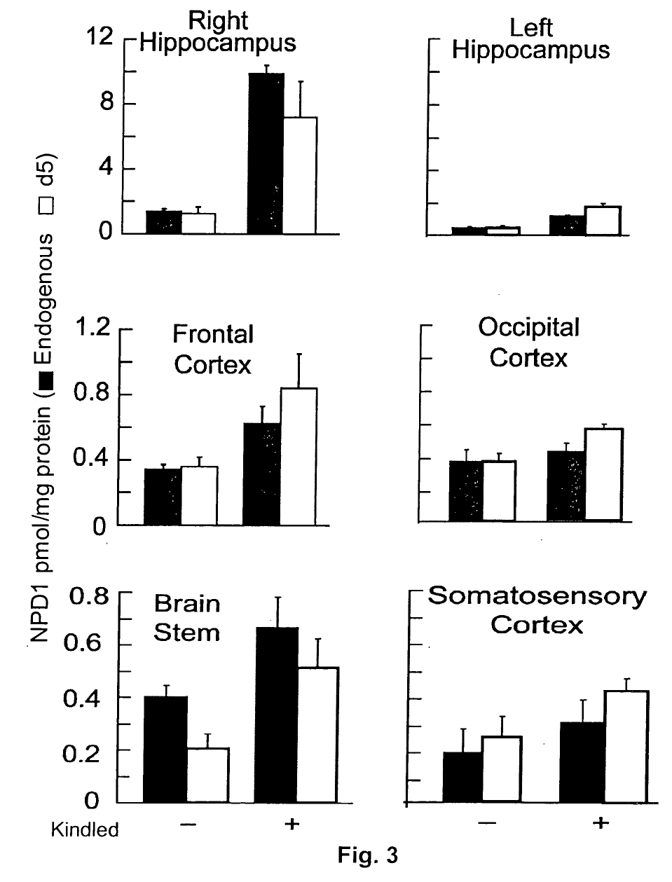

Mice again were implanted intraperitoneally with Alzet mini-pumps and were infused with a complex of human serum albumin and radiolabeled DHA (²H₅-DHA) at a rate of 1.68 μg/day/mouse. The mice were infused during the four days of kindling (500 nl/hr). In addition, the mice had a stimulating/recording electrode implanted in the dorsal hippocampus. By tandem LC-PDA-ESI-tandem MS-based lipidomic analysis, 10,17S-docosatriene (neuroprotectin D1) was measured from various regions of the brain. As shown in FIG. 3, neuroprotectin D1 was increased during kindling in all regions sampled. Both endogenous DHA and labeled DHA were metabolized to produce neuroprotectin D1.

The infusion of DHA-HSA had an inhibitory effect on the progression of kindling epileptogenesis. The cellular target and molecular pathways involved in DHA action, including the formation of neuroprotectin D1, may aid in developing novel neuroprotective therapeutic approaches in epileptogenesis.

The term “therapeutically effective amount” as used herein refers to an amount of the DHA-albumin complex sufficient either to inhibit or attenuate the symptoms of epilepsy to a statistically significant degree (p<0.05). The term “therapeutically effective amount” therefore includes, for example, an amount sufficient to decrease the number of epileptic events as measured by electroencephalography or as measured by Racine's score, and preferably to reduce such symptoms by at least 50%, and more preferably by at least 90%. The dosage ranges for the administration of DHA-albumin are those that produce the desired effect. A preferred dosage range is from about 0.3 mg to about 30 mg DHA-albumin complex per kilogram body weight. Generally, the dosage will vary with the age, weight, condition, and sex of the patient. A person of ordinary skill in the art, given the teachings of the present specification, may readily determine suitable dosage ranges. The dosage can be adjusted by the individual physician in the event of any contraindications. In any event, the effectiveness of treatment can be determined by monitoring the frequency of epileptic events by methods well known to those in the field and by methods taught by this Specification. Moreover, the DHA-albumin complex can be applied in pharmaceutically acceptable carriers known in the art. The application is preferably by injection or infusion.

The DHA-albumin complex may be administered to a patient by any suitable means, especially by parenteral. Parenteral infusions include intramuscular, intravenous, intraarterial, intraperitoneal or intravitreal administration. Additionally, the infusion could be directly into an organ, e.g., the brain. Injection of DHA-albumin may include the above infusions or may include intraperitonieal, intravitreal, direct injection into a blood vessel or into the cerebral spinal fluid. The DHA-albumin complex may also be administered transdermally, for example in the form of a slow-release subcutaneous implant, or orally in the form of capsules, powders, or granules. Although direct oral administration may cause some loss of activity, the DHA-albumin complex could be packaged in such a way to protect the active ingredient(s) from digestion by use of enteric coatings, capsules or other methods known in the art.

Pharmaceutically acceptable carrier preparations for parenteral administration include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, emulsions or suspensions, including saline, cerebral spinal fluid, and buffered media. Parenteral vehicles include sodium chloride solution, Ringer's dextrose, dextrose and sodium chloride, lactated Ringer's, or fixed oils. The DHA-albumin complex may be mixed with excipients that are pharmaceutically acceptable and are compatible. Suitable excipients include water, saline, dextrose, and glycerol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

The form may vary depending upon the route of administration. For example, compositions for injection may be provided in the form of an ampule, each containing a unit dose amount, or in the form of a container containing multiple doses. Direct injections into a blood vessel, or into the cerebral spinal fluid, or into the brain would be the most direct way to deliver the anti-epileptic complex to the target tissue.

Controlled delivery may be achieved by admixing the active ingredient with appropriate macromolecules, for example, polyesters, polyamino acids, polyvinyl pyrrolidone, ethylenevinylacetate, methylcellulose, carboxymethylcellulose, prolamine sulfate, or lactide/glycolide copolymers. The rate of release of DHA-albumin may be controlled by altering the concentration of the macromolecule.

Another method for controlling the duration of action comprises incorporating the DHA-albumin complex into particles of a polymeric substance such as a polyester, peptide, hydrogel, polylactide/glycolide copolymer, or ethylenevinylacetate copolymers. Alternatively, the DHA-albumin complex may be encapsulated in microcapsules prepared, for example, by coacervation techniques or by interfacial polymerization, for example, by the use of hydroxymethylcellulose or gelatin-microcapsules or poly(methylmethacrylate) microcapsules, respectively, or in a colloid drug delivery system. Colloidal dispersion systems include macromolecule complexes, nanocapsules, microspheres, beads, and lipid-based systems including oil-in-water emulsions, micelles, mixed micelles, and liposomes.

The present invention provides a method of treating or attenuating the symptoms of epilepsy, comprising administering to a patient at risk for epileptic seizures or a patient that has epileptic seizures, a therapeutically effective amount of DHA-albumin complex. The term “attenuate” refers to a decrease or lessening of the symptoms or signs of an epileptic seizure. The symptoms or signs that may be attenuated include those associated with an increase in neuronal activity in the brain during a seizure.

The complete disclosures of all references cited in this specification are hereby incorporated by reference. In the event of an otherwise irreconcilable conflict, however, the present specification shall control.

We claim:

1. A method to treat or attenuate symptoms associated with epileptic seizures, comprising administering to a patient susceptible to epilepsy a therapeutically effective amount of a complex comprising docosahexaenoic acid and albumin.

2. A method as in claim 1, wherein said administration is by intraperitoneal infusion or injection.

3. A method as in claim 1, wherein said administration is by intravenous injection or injection.

4. A method as in claim 1, wherein the therapeutically effective amount is from about 0.3 mg to about 30 mg DHA-albumin complex per kilogram body weight.

5. A method to reduce the incidence of epileptic seizures, comprising chronically administering to a patient susceptible to epilepsy a therapeutically effective amount of a complex comprising docosahexaenoic acid and albumin.

6. A method as in claim 5, wherein the chronic administration is by infusion intraperitoneally or intravascularly.

7. A method to alleviate symptoms from an epileptic seizure, comprising acutely administering to a patient having an epileptic seizure a therapeutically effective amount of a complex comprising docosahexaenoic acid and albumin.
